(19) United States
(12) Patent Application Publication
(10) Pub. No.: US 2012/0090611 A1
(43) Pub. Date: Apr. 19, 2012

(54) Systems And Methods For Controlling An Amount Of Oxygen In Blood Of A Ventilator Patient

(75) Inventors: Dan Graboi, Encinitas, CA (US); Peter Doyle, Vista, CA (US); Milenko Masic, San Diego, CA (US); Joseph Douglas Vandine, Manteca, CA (US)

(73) Assignee: Nellcor Puritan Bennett LLC, Boulder, CO (US)

(21) Appl. No.: 12/903,309

(22) Filed: Oct. 13, 2010

(51) Int. Cl. A61M 16/00

(52) U.S. Cl. 128/204.23

(57) ABSTRACT

This disclosure describes systems and methods for controlling blood oxygen saturation (SpO₂) or partial pressure of oxygen in arterial blood (PaO₂) of a patient being ventilated by a medical ventilator. The disclosure describes a novel approach of utilizing dynamic, real-time ventilator information in a closed-loop controller to determine the necessary FiO₂ and flow commands for the medical ventilator.

Medical ventilator systems have been long used to provide supplemental oxygen support to patients. These ventilators typically comprise a source of pressurized oxygen which is fluidly connected to the patient through a conduit. In some systems, the proper arterial oxygen saturation (SpO₂) is monitored via a pulse oximeter attached to the patient.

Some of these previously known medical ventilators attempt to automate the adjustment of fractional inspired oxygen (FiO₂) that is the oxygen fraction of the respiratory gas delivered to the patient, as a function of the patient's SpO₂. For instance, a ventilator system may adjust the FiO₂ in preset increments as a function of the value of the SpO₂, utilize fuzzy logic to automate the adjustment of FiO₂, and/or use empirically determined gain coefficients in a PID method (proportional, integral, derivative) to automate the adjustment of FiO₂. For example, if SpO₂ falls below or above a preset threshold in a patient, a controller may increase or decrease FiO₂ until the SpO₂ is above the threshold level.

While these previously known automated ventilation systems have effectively reduced the amount of required medical attention for the patient, they have not utilized any other available information to optimize or improve the control of monitored SpO₂ in a patient being ventilated.

### SUMMARY

This disclosure describes systems and methods for controlling blood oxygen saturation (SpO₂) or partial pressure of oxygen in arterial blood (PaO₂) of a patient being ventilated by a medical ventilator. The disclosure describes a novel approach of utilizing dynamic, real-time ventilator information in a closed-loop controller to determine the necessary FiO₂ and flow commands for the medical ventilator.

In part, this disclosure describes a method for controlling an amount of oxygen in blood in a patient being ventilated by a medical ventilator. The method includes:

(a) monitoring an amount of oxygen in blood in a patient during ventilation on the medical ventilator;

(b) monitoring privileged ventilator information, the privileged ventilator information is flow rate, compliance of patient circuit, and minute volume; and

(c) controlling at least one of a specific oxygen percentage in a gas mixture supplied by the ventilator to the patient and a gas flow rate of the gas mixture supplied by the ventilator to the patient during ventilation based on the monitored amount of oxygen in the blood of the patient and the monitored privileged ventilator information.

Another aspect of this disclosure describes a method for controlling an amount of oxygen in blood in a patient being ventilated by a medical ventilator. The method includes:

(a) monitoring an amount of oxygen in blood in a patient being ventilated by a medical ventilator;

(b) monitoring privileged ventilator information, the privileged ventilator information is flow rate, compliance of patient circuit, and minute volume;

(c) detecting apnea in the patient based on the monitored amount of oxygen in the blood in the patient and the monitored privileged ventilator information; and

(d) sending a few small breaths through the ventilator circuit to stimulate breathing in the patient.

Additionally, this disclosure describes a medical ventilator system. The medical ventilator system includes:

(a) means for repeatedly monitoring an amount of oxygen in blood in a patient during ventilation on the medical ventilator;

(b) means for repeatedly monitoring privileged ventilator information, wherein the ventilator privileged information comprises flow rate, compliance of a patient circuit, minute volume, and ideal body weight; and

(c) means for determining if a change in at least one of an oxygen percentage or flow rate is necessary based on the monitored amount of oxygen in the blood in the patient and the monitored privileged ventilator information; and

(d) means for adjusting at least one of the oxygen percentage in a gas mixture supplied by the ventilator to the patient and the gas flow rate of the gas mixture supplied by the ventilator to the patient during ventilation based on the monitored amount of oxygen in the blood in the patient and the monitored privileged ventilator information

In another aspect, this disclosure describes a non-transitory computer-readable medium having computer-executable instructions for performing a method for controlling an amount of oxygen in blood in a patient being ventilated by a medical ventilator. The method includes:

(a) repeatedly monitoring an amount of oxygen in blood in a patient during ventilation on the medical ventilator;

(b) repeatedly monitoring privileged ventilator information, the privileged information comprises flow rate, compliance of a patient circuit, and minute volume;

(c) determining that a change in at least one of an oxygen percentage or flow rate is necessary based on the monitored amount of oxygen in the blood in the patient and the monitored privileged ventilator information; and

(d) adjusting at least one of the oxygen percentage in a gas mixture supplied by the ventilator to the patient and the gas flow rate of the gas mixture supplied by the ventilator to the patient during ventilation based on the monitored amount of oxygen in the blood in the patient and the monitored privileged ventilator information.

The disclosure further describes a medical ventilator system that includes: a processor; a patient circuit; an oximeter connected to a patient being ventilated by the medical ventilation system and controlled by the processor; and an SpO₂ controller in communication with the processor and the oximeter. The privileged ventilator information is flow rate, compliance of a patient circuit, minute volume, and ideal body weight (IBW). The oximeter is adapted to monitor a blood oxygen saturation level of the patient during ventilation by the medical ventilator system. The SpO₂ controller is adapted receive the monitored blood oxygen saturation level from the oximeter, is adapted to receive privileged ventilator information from the processor, and is adapted to control at least one of a specific oxygen percentage and a flow rate of a gas mixture supplied to the patient during ventilation by the medical ventilator system based on the monitored blood oxygen saturation level of the patient and the privileged ventilator information.

Additionally, the disclosure further describes a medical ventilator that includes: a processor; a patient circuit; a blood gas monitor connected to a patient being ventilated by a medical ventilator system and controlled by the processor, the blood gas monitor is adapted to monitor a partial pressure of oxygen in the patient during ventilation by the medical ventilator system; and a PaO₂ controller in communication with the processor and the blood gas monitor and adapted to receive the monitored partial pressure of oxygen in the patient from the blood gas monitor, adapted to receive privileged ventilator information from the processor, and adapted to control at least one of a specific oxygen percentage and a flow rate of a gas mixture supplied to the patient during ventilation by the medical ventilator system based on the monitored partial pressure of oxygen in the patient and the privileged ventilator information. The privileged ventilator information is flow rate, compliance of a patient circuit, minute volume, and ideal body weight.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments systems and methods described below and are not meant to limit the scope of the invention in any manner, which scope shall be based on the claims appended hereto.

FIG. 1 illustrates an embodiment of a ventilator connected to a human patient.

FIG. 2 illustrates an embodiment of SpO₂ controller operatively coupled with a medical ventilator and an oximeter.

FIG. 3 illustrates an embodiment of a method for controlling blood oxygen saturation of a patient being ventilated by a medical ventilator.

FIG. 4 illustrates an embodiment of a method for controlling blood oxygen saturation of a patient being ventilated by a medical ventilator.

FIG. 5 illustrates a graph of a monitored oxygen percentage of a gas mixture at a patient wye in response to an executed ventilator mix (FiO₂) change per second from a change in mixture for eight varying series run on a ventilator ventilating a simulated neonate.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. The reader will understand that the technology described in the context of a medical ventilator for human patients could be adapted for use with other systems such as ventilators for non-human patients and general gas transport systems in which sensor tubes in challenging environments may require periodic or occasional purging.

Medical ventilators are used to provide a breathing gas to a patient who may otherwise be unable to breathe sufficiently. In modern medical facilities, pressurized air and oxygen sources are often available from wall outlets. Accordingly, ventilators may provide flow regulating valves connected to centralized sources of pressurized air and pressurized oxygen. The flow regulating valves function to regulate flow so that respiratory gas having a desired concentration of oxygen is supplied to the patient at desired pressures/volumes and rates. Ventilators capable of operating independently of external sources of pressurized air are also available. As utilized herein a “gas mixture” includes a pure gas and/or a mixture of pure gases.

While operating a ventilator, it is desirable to control the percentage of oxygen in the gas supplied by the ventilator to the patient, referred to as the fractional inspired oxygen or FiO₂. Further, it is desirable to monitor the oxygen saturation level of blood in a patient. The oxygen saturation level may be monitored by any suitable method, now known or later developed, and specifically including by pulse oximetry or by direct measurement. For convenience, the oxygen saturation level of a patient shall be referred to as the “SpO₂ level” even though that nomenclature is normally used to indicate the oxygen saturation level as monitored by a pulse oximeter. Likewise, embodiments described herein illustrate the use of pulse oximeter and the reader should keep in mind that other types of oximeters could alternatively be used.

The adjustment of FiO₂ levels based on SpO₂ levels may be referred to as “closed loop” control or “closed loop” systems to indicate the ability to automatically control the FiO₂ levels. For closed loop ventilators it is desirable to provide for a closed loop controller with better stability and response time. Accordingly, a closed loop controller was designed that utilizes dynamic real-time information from a ventilator to provide for stability and better response time. The dynamic real-time information or “privileged information” from the ventilator is available at all times and includes information such as ventilator parameters, patient data, sensor readings, and inputted data. In one embodiment, the ventilator privileged information includes the instantaneous flow being supplied by the ventilator and knowledge of the compliance of the patient circuit.

A closed loop controller with access to such privileged information can utilize this information to better determine a time for a change in oxygen percent for delivery from the ventilator to the lungs of the patient. As the flow decreases, the closed loop controller can modify parameters, such as “washout” time for the inspiratory limb of the patient circuit to change from one percentage of oxygen in the gas mixture to another percentage. As used herein the term “washout time” refers to the amount of time necessary for an oxygen percentage setting change to be realized in the breathing circuit adjacent to the patient interface, such as the patient wye. In an alternative embodiment, if apnea is detected, the closed loop controller can deliver a few small breaths. The few small breaths will help stimulate breathing in apneic patients, such as neonates, and help avoid the over-delivery of oxygen. The proposed controller could also take advantage of privileged ventilator information such as flow rate, ideal body weight, gas mixture, and/or circuit compliance to provide for improved performance.

For example, the gain coefficients of a proportional-integral-derivative (PID) controller can be changed depending on flow rate and compliance, thus helping to prevent overshoot, undershoot, and oscillation of SpO₂ while providing improved speed of control as compared to a controller not so equipped. As used herein the term “PID controller” includes proportional-integral (PI), proportional (P), integral (I), proportional-derivative (PD), integral derivative (ID), and derivative (D) controllers because the value of a parameter (P, I, and/or D) may be zero. Furthermore, knowledge of flow rate and patient circuit compliance can be used to implement a “fast washout” cycle by momentarily increasing flow to an appropriate higher value while opening both inspiratory and expiratory valves. Such action can be performed without detriment to patient ventilation. This fast washout cycle may decrease washout time by at least 25% and in some instances by at least 75% thereby decreasing the amount of time it takes for the patient to receive an oxygen setting change. Additional ventilator privileged information includes, but it is not limited to minute volume, which can be used to estimate lung washout time, and ideal body weight (IBW) of the patient, which can be used to estimate circulatory time and lung washout time. Again such information can be utilized to further improve controller performance. It is understood by a person of skill in the art that any suitable ventilator information and combinations of information for aiding in the function of a closed loop SpO₂ controller may be accessed and/or utilized by a closed-loop SpO₂ controller.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications. In this regard, any number of the features of the different embodiments described herein may be combined into single-component or multiple-component embodiments, and alternative embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

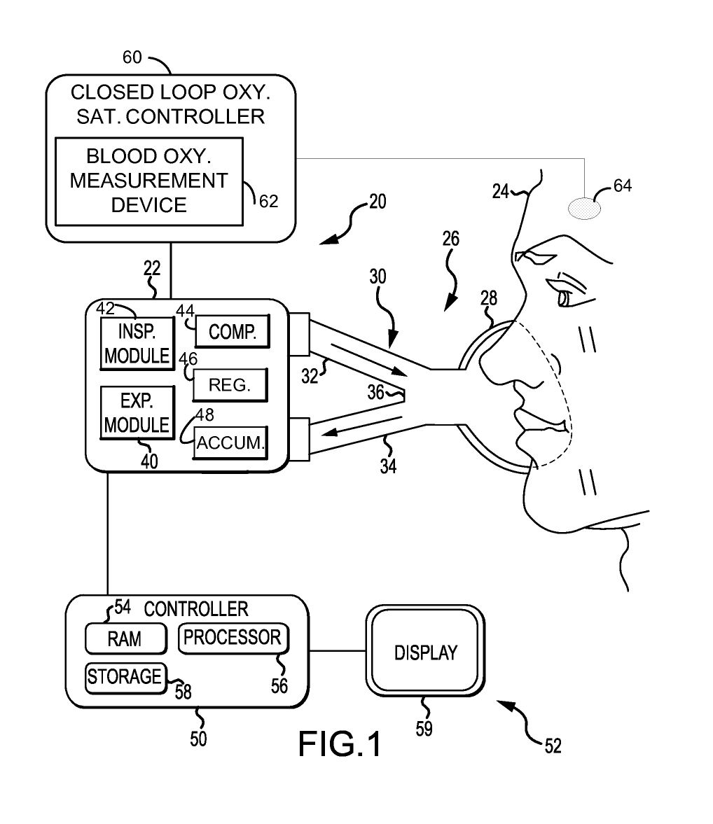

FIG. 1 illustrates an embodiment of a ventilator 20 connected to a human patient 24. Ventilator 20 includes a pneumatic system 22 (also referred to as a pressure generating system 22) for circulating breathing gases to and from patient 24 via the ventilation tubing system 26, which couples the patient 24 to the pneumatic system 22 via physical patient interface 28 and ventilator circuit 30. Ventilator 20 also includes a closed loop oxygen saturation controller (SpO₂ controller) 60 including an oximeter 62 for measuring the SpO₂ of patient 24 connected to the ventilator 20 during ventilation.

Ventilator circuit 30 could be a two-limb or one-limb circuit 30 for carrying gas to and from the patient 24. In a two-limb embodiment as shown, a wye fitting 36 may be provided as shown to couple the patient interface 28 to the inspiratory limb 32 and the expiratory limb 34 of the circuit 30.

The present description contemplates that the patient interface 28 may be invasive or non-invasive, and of any configuration suitable for communicating a flow of breathing gas from the patient circuit 30 to an airway of the patient 24. Examples of suitable patient interface 28 devices include a nasal mask, nasal/oral mask (which is shown in FIG. 1), nasal prong, full-face mask, tracheal tube, endrotracheal tube, nasal pillow, etc.

Pneumatic system 22 may be configured in a variety of ways. In the present example, system 22 includes an expiratory module 40 coupled with an expiratory limb 34 and an inspiratory module 42 coupled with an inspiratory limb 32. Compressor 44 or another source or sources of pressurized gas (e.g., pressured air and/or oxygen) that provide gas supply is controlled through the use of one or more gas regulators or flow valves 46. Further, the gas concentrations are mixed and/or stored in a chamber of a gas accumulator 48 at a high pressure to improve the control of delivery of respiratory gas to the ventilator circuit 30. The inspiratory module 42 is coupled to the compressor 44, the gas regulator or flow valve 46, and accumulator 48 to control the source of pressurized breathing gas for ventilator support via inspiratory limb 32.

The pneumatic system 22 may include a variety of other components, including sources for pressurized air and/or oxygen, mixing modules, valves, sensors, tubing, filters, etc.

A closed loop SpO₂ controller 60 is operatively coupled with the pneumatic system 22. The closed loop SpO₂ controller 60 may include memory, one or more processors, storage, and/or other components of the type commonly found in command and control computing devices. In the embodiment shown, the closed loop SpO₂ controller 60 further includes an oximeter 62. The oximeter 62 is connected to a patient oximeter sensor 64. In an alternative embodiment, the oximeter 62 is part of the ventilator system 20 or the pneumatic system 22. In another embodiment, the oximeter 62 is a completely separate and independent component from the ventilator 20 and the SpO₂ controller 60.

The oximeter 62 monitors a blood oxygen saturation level of the patient 24 based on the patient readings taken by the patient oximeter sensor 64 during ventilation of the patient 24 by the ventilator 20. The oximeter sends the monitored oxygen gas saturation level of the blood of the patient 24 to the SpO₂ controller 60. Further, dynamic, real time, and/or privileged ventilator information is sent from the ventilator 20 to the SpO₂ controller 60. In one embodiment, the privileged ventilator information is sent by the controller 50 from the ventilator 20 to the SpO₂ controller 60. The SpO₂ controller 60 utilizes the blood gas oxygen saturation level along with the dynamic, real time ventilator information to determine a desired fractional inspired oxygen percentage and a desired gas flow rate. In one embodiment, the SpO₂ controller 60 utilizes preset increments as a function of the value of the SpO₂ and one or more parameters obtained from the ventilator privileged information. In another embodiment, the SpO₂ controller 60 utilizes fuzzy logic to automate the adjustment of FiO₂ based on the SpO₂ patient measurements and one or more parameters obtained from the ventilator privileged information. In an alternative embodiment, SpO₂ controller 60 utilizes empirically determined or computed gain coefficients based on the SpO₂ patient measurements and one or more parameters obtained from the ventilator privileged information in a proportional-integral-derivative (PID) method to automate the adjustment of FiO₂. For example, if SpO₂ falls below or above a preset threshold in a patient 24 with an ideal body weight in a specific range, SpO₂ controller 60 may increase or decrease FiO₂ in preset increments until the SpO₂ is above the threshold level. In an alternative embodiment, if apnea is detected, the SpO₂ controller 60 may deliver a few small breaths. The few small breaths will help stimulate breathing in apneic patients, such as neonates, and help avoid the over-delivery of oxygen.

The SPO₂ controller 60 sends a command to the ventilator 20 causing the ventilator 20 to implement the desired fractional inspired oxygen percentage and the desired gas flow rate. In one embodiment, the SpO₂ controller 60 sends a command to the controller 50 of the ventilator 20 and the controller 50 causes the ventilator 20 to implement the desired fractional inspired oxygen percentage and the desired gas flow rate.

The privileged ventilator information includes pre-set ventilator parameters, inputted parameters, sensor readings, and/or monitored patient parameters. In one embodiment, the dynamic, real time ventilator information includes at least one of a respiratory rate, a tidal volume, a compliance of the patient circuit, or ideal body weight.

Controller 50 is operatively coupled with pneumatic system 22, closed loop SpO₂ controller 60, signal measurement and acquisition systems, and an operator interface 52, which may be provided to enable an operator to interact with the ventilator 20 (e.g., change ventilator settings, select operational modes, view monitored parameters, etc.). Controller 50 may include memory 54, one or more processors 56, storage 58, and/or other components of the type commonly found in command and control computing devices.

The memory 54 is non-transitory computer-readable storage media that stores software that is executed by the processor 56 and which controls the operation of the ventilator 20. In an embodiment, the memory 54 comprises one or more solid-state storage devices such as flash memory chips. In an alternative embodiment, the memory 54 may be mass storage connected to the processor 56 through a mass storage controller (not shown) and a communications bus (not shown). Although the description of non-transitory computer-readable media contained herein refers to a solid-state storage, it should be appreciated by those skilled in the art that non-transitory computer-readable storage media can be any available media that can be accessed by the processor 56. Non-transitory computer-readable storage media includes volatile and non-volatile, removable and non-removable media implemented in any method or technology for storage of information such as computer-readable instructions, data structures, program modules or other data. Non-transitory computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 56.

The controller 50 issues commands to pneumatic system 22 in order to control the breathing assistance provided to the patient 24 by the ventilator 20. The specific commands may be based on inputs received from patient 24, pneumatic system 22 and sensors, operator interface 52 and/or other components of the ventilator 20. In the depicted example, operator interface 52 includes a display 59 that is touch-sensitive, enabling the display 59 to serve both as an input user/operator interface and an output device. The display 59 can display any type of ventilation information, such as sensor readings, parameters, commands, alarms, warnings, and smart prompts (i.e., ventilator determined operator suggestions).

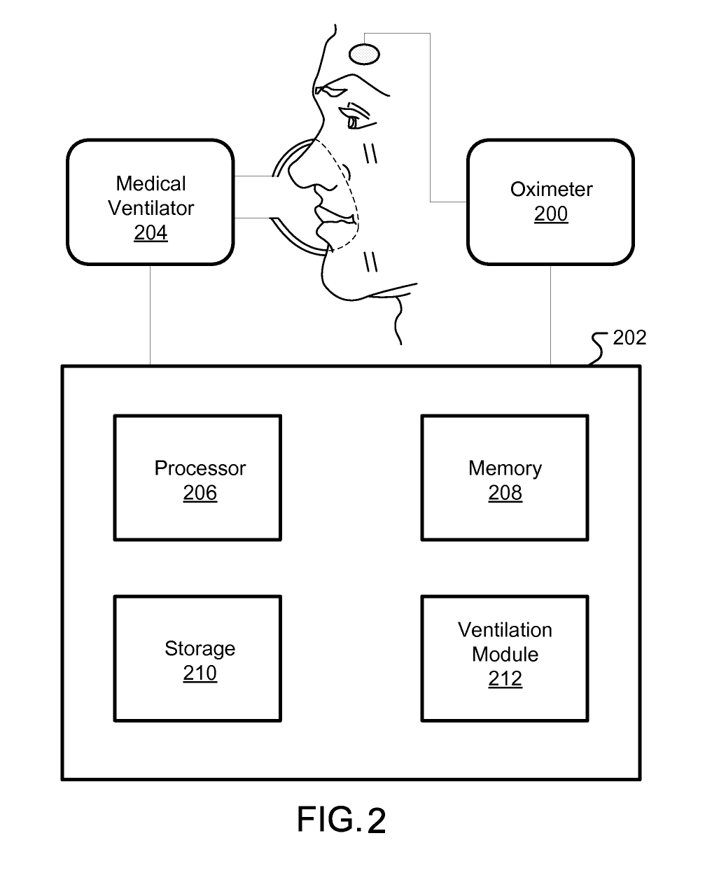

FIG. 2 illustrates an embodiment of a closed loop SpO₂ controller 202 operatively coupled with a medical ventilator 204 and an oximeter 200. As illustrated in FIG. 2, SpO₂ controller 202 may be a separate component from the ventilator 204 and the oximeter 200. In an alternative embodiment, not shown, SpO₂ controller 202 may be a part of the ventilator 204.

The oximeter 200 has a sensor attached to a patient for determining the arterial oxygen saturation of a patient being ventilated by a medical ventilator 204. The oximeter readings are sent to the SpO₂ controller 202.

SpO₂ controller 202 may include memory 208, one or more processors 206, storage 210, and/or other components of the type commonly found in command and control computing devices.

The memory 208 is non-transitory computer-readable storage media that stores software that is executed by the processor 206 and which controls the gas flow rate of the gas mixture and oxygen concentration of the gas mixture delivered to a patient by the ventilator 204. In an embodiment, the memory 208 comprises one or more solid-state storage devices such as flash memory chips. In an alternative embodiment, the memory 208 may be mass storage connected to the processor 206 through a mass storage controller (not shown) and a communications bus (not shown). Although the description of non-transitory computer-readable media contained herein refers to a solid-state storage, it should be appreciated by those skilled in the art that non-transitory computer-readable storage media can be any available media that can be accessed by the processor 206. Non-transitory computer-readable storage media includes volatile and non-volatile, removable and non-removable media implemented in any method or technology for storage of information such as computer-readable instructions, data structures, program modules or other data. Non-transitory computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 206.

The SpO₂ controller 202 issues commands to the ventilator 204 or to the pneumatic system of the ventilator 204 in order to control the flow rate of the gas mixture and the oxygen percentage of the gas mixture provided to the patient by the ventilator 204. The specific commands may be based on the blood gas oxygen saturation level of the patient and inputs received from patient 24, pneumatic system and sensors, operator interface and/or other ventilator privileged information of the ventilator 204. In the depicted example, the ventilator 204 may further include a display that is touch-sensitive, enabling the display to serve both as an input user interface and an output device. The display can display any type of ventilation, oximeter, or SpO₂ controller information, such as sensor readings, parameters, commands, alarms, warnings, and smart prompts (i.e., ventilator determined operator suggestions).

SpO₂ controller 202 can utilize ventilator privileged information to better determine a time for a change in oxygen percent for delivery from the ventilator to the lungs of the patient. As the flow decreases, the SpO₂ controller 202 can send commands to the ventilator 204 to modify parameters, such as “washout” time for the Inspiratory limb of the patient circuit to change from one percentage of oxygen in the gas mixture to another percentage. SpO₂ controller 202 can also take advantage of privileged ventilator knowledge to provide for improved performance. In one embodiment, the closed loop SpO₂ controller 202 utilizes at least one of flow rate, ideal body weight (IBW), gas mixture, and/or circuit compliance to provide for improved performance.

In the embodiment shown, the SpO₂ controller 202 further includes a ventilation module 212. The ventilation module 212 includes the logic, preset parameters, functions, and/or equations for determining how to control the flow rate of the gas mixture and the oxygen percentage of the gas mixture provided to the patient by the ventilator 204. In one embodiment, the ventilation module 212 utilizes preset increments as a function of the value of the SpO₂ and one or more parameters obtained from the ventilator privileged information. In another embodiment, ventilation module 212 utilizes fuzzy logic to automate the adjustment of FiO₂ based on the SpO₂ patient measurements and one or more parameters obtained from the ventilator privileged information. In an alternative embodiment, ventilation module 212 utilizes empirically determined gain coefficients based on the SpO₂ patient measurements and one or more parameters obtained from the ventilator privileged information in a PID method (proportional, integral, and derivative) to automate the adjustment of FiO₂.

For example, if SpO₂ falls below a preset low threshold or above a preset high threshold in a patient with an ideal body weight in a specific range, the ventilation module 212 of the SpO₂ controller 202 may send a command to the ventilator to increase or decrease FiO₂ in preset increments until the SpO₂ is between the preset high and low threshold levels. In another example, the gain coefficients of a ventilation module 212 utilizing a PID method can be changed depending on flow rate and compliance, thus helping to prevent overshoot, undershoot, and oscillation of SpO₂ while providing improved speed of control as compared to a controller without privileged ventilator information. Furthermore, knowledge of flow rate and patient circuit compliance can be used to implement a “fast washout” cycle by momentarily increasing flow to an appropriate higher value while opening both inspiratory and expiratory valves. Such action can be performed without detriment to patient ventilation. This fast “washout cycle” may decrease washout time by at least 25% and in some instances by at least 75% and thereby decreases the amount of time it takes for an oxygen setting change to reach a patient. Ventilator privileged information, such as minute volume, which can be used to estimate lung washout time, and ideal body weight (IBW) of the patient, can be used to estimate circulatory time and lung washout time. Knowledge of circulatory time can improve overshoot and undershoot performance of the controller 202 when changing the oxygen percentage in the gas mixture. In an alternative embodiment, if apnea is detected, the SpO₂ controller 202 can deliver a few small breaths. The few small breaths will help stimulate breathing in apneic patients, such as neonates, and help avoid the over-delivery of oxygen. Again such information can be utilized to further improve controller performance. It is understood by a person of skill in the art that any suitable ventilator information and combinations of information for aiding in the function of a closed loop controller may be accessed and/or utilized by a closed-loop controller.

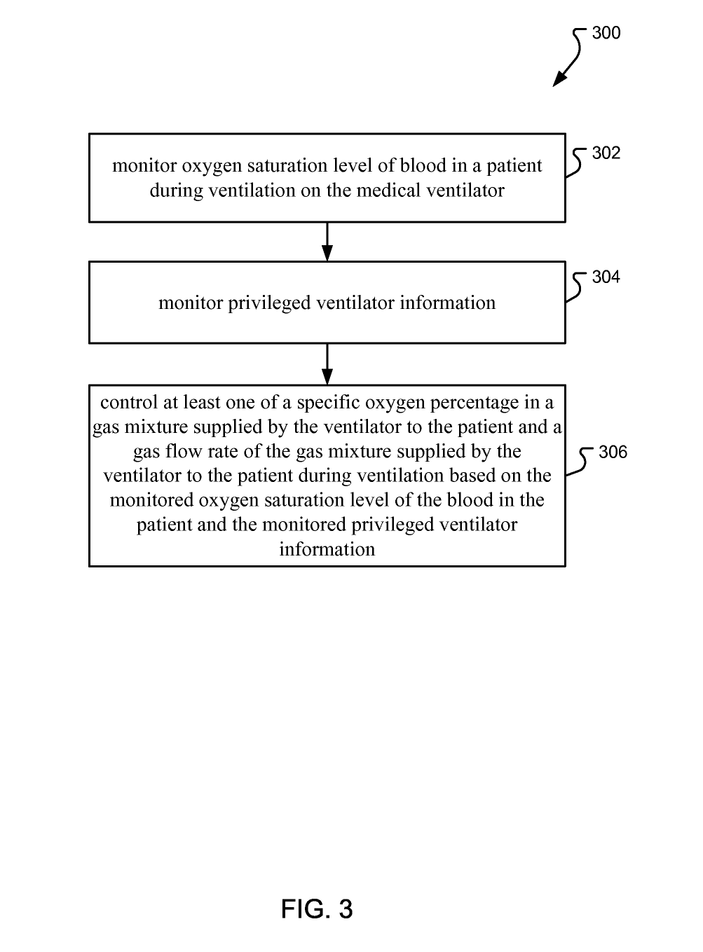

FIG. 3 illustrates a method for controlling blood oxygen saturation of a patient being ventilated by a medical ventilator, 300. Accordingly, method 300 monitors oxygen saturation level of blood in a patient during ventilation on the medical ventilator, 302. In one embodiment, step 302 is performed by an oximeter. In one embodiment, step 302 monitors blood oxygen saturation levels continuously. In another embodiment, step 302 monitors blood oxygen saturation levels upon command. In a further embodiment, step 302 monitors blood oxygen saturation levels in preset or user determined time intervals.

Method 300 monitors privileged ventilator information, 304. Privileged ventilator information includes past and current or real-time information from a ventilator. The privileged information is available at all times from the ventilator and includes information such as ventilator parameters, patient data, sensor readings, and inputted data. In one embodiment, the ventilator privileged information includes the instantaneous flow being supplied by the ventilator and knowledge of the compliance of the patient circuit. Additional ventilator privileged information includes, but are not limited to minute volume and ideal body weight (IBW) of the patient. It is understood by a person of skill in the art that any suitable ventilator information and combinations of information for aiding in the method for controlling blood oxygen saturation of a patient being ventilated by a medical ventilator may be accessed and/or utilized by method 300.

Method 300 controls at least one of a specific oxygen percentage in a gas mixture supplied by the ventilator to the patient and a gas flow rate of the gas mixture supplied by the ventilator to the patient during ventilation based on the monitored oxygen saturation level of the blood of the patient and the monitored privileged ventilator information 306. For instance, as the flow decreases, method 300 can modify parameters, such as “washout” time for the inspiratory limb of the patient circuit to change from one percentage of oxygen in the gas mixture to another percentage. In an alternative embodiment, if apnea is detected, method 300 can have a few small breaths delivered. The few small breaths will help stimulate breathing in apneic patient, such as neonates, and help avoid the over-delivery of oxygen. In another embodiment, if SpO₂ falls below a preset low threshold or above a preset high threshold in a patient with an ideal body weight in a specific range, method 300 can increase or decrease FiO₂ in preset increments until the SpO₂ is between the high and low threshold levels. In another example, the gain coefficients of a ventilation module utilizing a PID method can be adjusted by method 300 depending on flow rate and compliance, thus helping to prevent overshoot, undershoot, and oscillation of SpO₂. Based on flow rate and patient circuit compliance, method 300 can implement a “fast washout” cycle by momentarily increasing flow to an appropriate higher value while opening both inspiratory and expiratory valves. This fast washout cycle may decrease washout time by 25% and in some instances by as much as 75% and thereby decreases the amount of time it takes for the patient to receive an oxygen setting change. Based on ventilator privileged information such as minute volume and ideal body weight (IBW), method 300 can estimate lung washout time. Based on IBW of the patient, method 300 can estimate circulatory time. Knowledge of circulatory time can improve overshoot and undershoot for changes in the oxygen percentage in the gas mixture.

In another embodiment, a SpO₂ controller for a medical ventilator may comprise a microprocessor continuously receiving a monitored oxygen saturation level of blood in a patient during ventilation by a medical ventilator and continuously receiving privileged ventilator information from the medical ventilator and adapted to utilize the received privileged ventilator information and the received monitored gas oxygen saturation level of the blood of the patient during ventilation by the medical ventilator to control at least one of a specific oxygen percentage and a flow rate of a gas mixture supplied to the patient by the medical ventilator during ventilation.

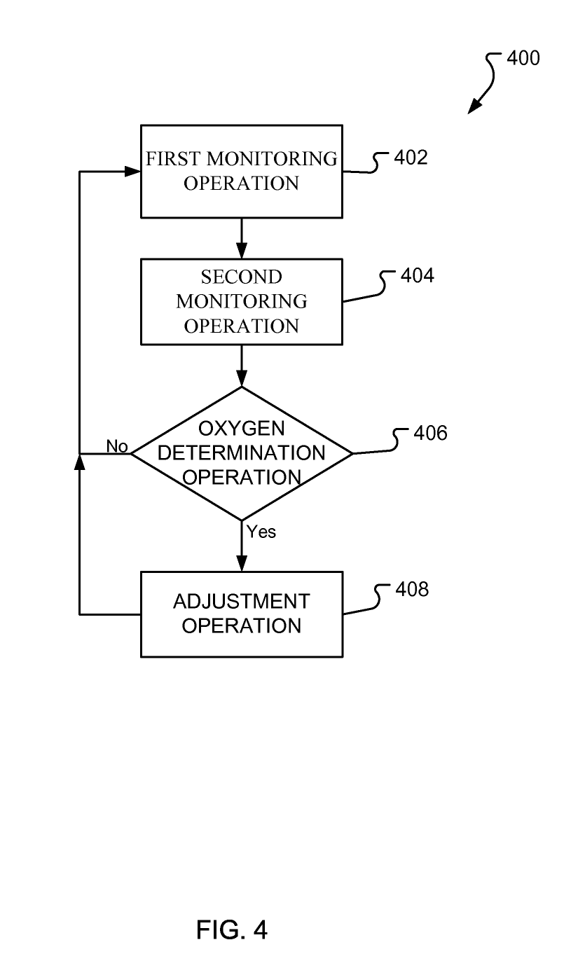

In a further embodiment, as illustrated in FIG. 4, a non-transitory computer-readable medium having computer-executable instructions for performing a method 400 for controlling blood oxygen saturation of a patient being ventilated by a medical ventilator is disclosed. Method 400 includes a first monitoring operation 402 that repeatedly monitors oxygen saturation level of blood in a patient during ventilation on the medical ventilator. Method 400 further includes a second monitoring operation 404 for repeatedly monitoring privileged ventilator information. The ventilator privileged information includes flow rate, compliance of a patient circuit, estimated patient lung compliance, dynamic gas mixture composition, minute volume, and estimated circulatory time. In one embodiment, the ventilator privileged information also includes ideal body weight (IBW).

As illustrated in FIG. 4, method 400 performs an oxygen determination operation 406 for determining if a change in at least one of an oxygen percentage or flow rate is necessary based on the monitored oxygen saturation level of the blood of the patient and the monitored privileged ventilator information. If oxygen determination operation 406 determines that a change in at least one of the oxygen percentage or flow rate is necessary based on the monitored oxygen saturation level of the blood of the patient and the monitored privileged ventilator information, then oxygen determination operation 406 selects to perform adjustment operation 408. If oxygen determination operation 406 determines that a change in at least one of the oxygen percentage or flow rate is not necessary based on the monitored oxygen saturation level of the blood of the patient and the monitored privileged ventilator information, then oxygen determination operation 406 selects to perform first monitoring operation 402.

The adjustment operation 408 of method 400 adjusts at least one of the oxygen percentage in a gas mixture supplied by the ventilator to the patient and the gas flow rate of the gas mixture supplied by the ventilator to the patient during ventilation based on the monitored oxygen saturation level of the blood of the patient and the monitored privileged ventilator information. For instance, as the flow decreases, method 400 can modify parameters, such as “washout” time for the inspiratory limb of the patient circuit during a change from one percentage of oxygen in the gas mixture to another percentage. In an alternative embodiment, if apnea is detected, method 400 can have a few small breaths delivered. The few small breaths will help stimulate breathing in apneic patients, such as neonates, and help avoid the over-delivery of oxygen. In another embodiment, if SpO₂ falls below a preset low threshold or above a preset high threshold in a patient with an ideal body weight in a specific range, method 400 can increase or decrease FiO₂ in preset increments until the SpO₂ is between the preset high and low threshold levels. In another example, the gain coefficients of a ventilation module utilizing a PID method can be adjusted by method 400 depending on flow rate and compliance, thus helping to prevent overshoot, undershoot, and oscillation of SpO₂. Based on flow rate and patient circuit compliance, method 400 can implement a “fast washout” cycle by momentarily increasing flow to an appropriate higher value while opening both inspiratory and expiratory valves. This fast washout cycle may decrease washout time by at least 25% and in some instances by at least 75% and thereby decreases the amount of time it takes for the patient to receive an oxygen setting change. Based on ventilator privileged information, such as minute volume, method 400 can estimate lung washout time. Based on ideal body weight (IBW) of the patient, method 400 can estimate circulatory time. Knowledge of circulatory time can improve overshoot and undershoot for changes in the oxygen percentage in the gas mixture.

In one embodiment, after method 400 performs the adjustment operation 408, method 400 performs first monitoring operation 402 again.

In one embodiment, a medical ventilator system includes means for repeatedly monitoring oxygen saturation level of blood in a patient during ventilation on the medical ventilator. Examples of these means are described in the description of FIG. 1 above. In an embodiment, a medical ventilator system includes means for repeatedly monitoring privileged ventilator information. The ventilator privileged information includes flow rate, compliance of a patient circuit, minute volume, and ideal body weight. Examples of means for repeatedly monitoring privileged ventilator information are also disclosed in the description in FIG. 1 above. In another embodiment, a medical ventilator system includes means for determining if a change in at least one of an oxygen percentage or flow rate is necessary based on the monitored oxygen saturation level of the blood of the patient and the monitored privileged ventilator information. The description of FIG. 1 above provides examples of suitable means for determining if a change in at least one of an oxygen percentage or flow rate is necessary. Further, in an embodiment, a medical ventilator system, includes means for adjusting at least one of the oxygen percentage in a gas mixture supplied by the ventilator to the patient and the gas flow rate of the gas mixture supplied by the ventilator to the patient during ventilation based on the monitored oxygen saturation level of the blood of the patient and the monitored privileged ventilator information. The description of FIG. 1 above also provides examples of suitable means for adjusting at least one of the oxygen percentage in a gas mixture supplied by the ventilator to the patient and the gas flow rate of the gas mixture supplied by the ventilator to the patient. The example means shown in FIG. 1 and described above are exemplary only and not meant to limit the description of this example and method 400.

In an alternative embodiment, all of the methods and systems described above and illustrated in FIGS. 1-4 may determine the level of oxygen in the blood of the patient by measuring the partial pressure of arterial oxygen (PaO₂) instead of the oxygen saturation level (SpO₂). Accordingly, everywhere in the description above and in FIGS. 1-4 where an oximeter is utilized, in this embodiment, a blood gas monitor is utilized instead. Further, everywhere in the description above and in FIGS. 1-4 where a SpO₂ is utilized, in this embodiment, PaO₂ is utilized instead.

### Example 1

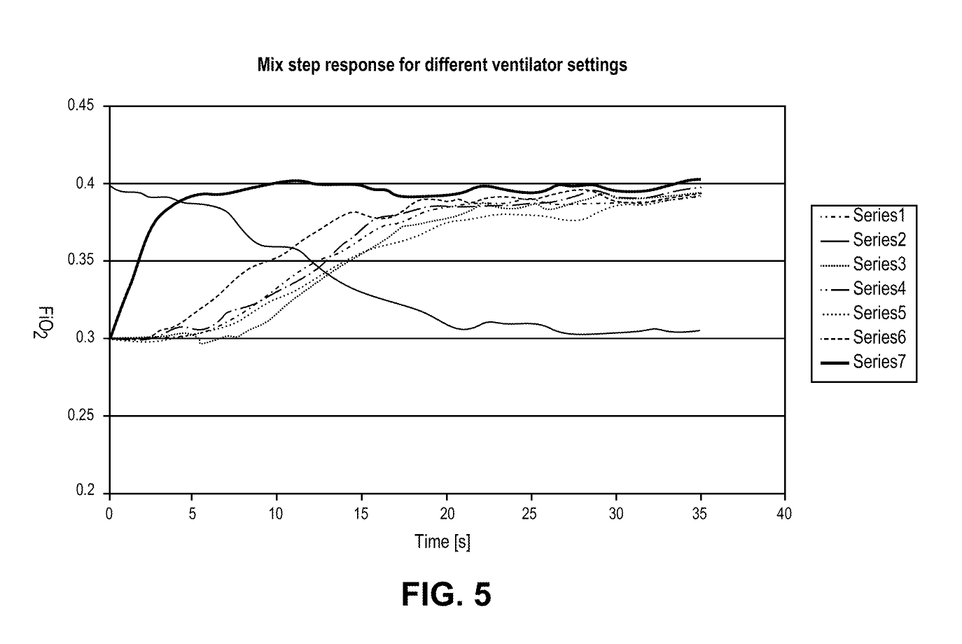

Eight different data series involving a change in oxygen percentage were run on a ventilator ventilating a simulated neonate. The concentrations of oxygen at the patient wye were recorded with an O₂ analyzer from the time of execution of the oxygen percentage change to 35 seconds from the execution of the change. Table 1 below lists the parameters used for each series and the measured oxygen percentage monitored by the O₂ analyzer at the patient wye for every second from 0 to 35 seconds. The data listed in Table 1 and graphed in FIG. 5 has been corrected for O₂ analyzer latency. FIG. 5 graphs the measured oxygen percentages listed in Table 1 taken by the O₂ analyzer at the wye for 35 seconds.

As illustrated in FIG. 5, Series 1, 3, 4 and 5, where the flow rate is 0.5 Li/min, show that it takes about 25 to 30 seconds for an oxygen setting change from 30% to 40% to be realized at the patient wye in ventilator during the ventilation of a simulated neonate. Series 6 and 7, where the flow rates are 1 Li/min and 5 Li/min respectively, show faster times for the oxygen setting change to be realized at the patient wye. Series 2 shows the result of changing the oxygen setting from 40% to 30% where the flow rate is 0.5 Li/min. FIG. 5 illustrates that a significant amount of time passes (i.e. over 20 seconds) before an executed change in oxygen percentage reaches the neonate. Accordingly, increasing base flow during exhalation or utilizing a “fast washout” cycle to reduce washout time improves a closed loop controller and reduces the amount of time it takes for a change in oxygen percentage to reach a patient.

TABLE 1
Response time for a change in oxygen percentage at different ventilator settings.

| | | | | | | Series 6 | Series 7 |
|---|---|---|---|---|---|---|---|
| | Series 1 | Ser 2 | Series 3 | Series 4 | Series 5 | BR = 20; | BR = 20; |
| | BR = 20; | BR = 20; | BR = 20; | BR = 20; | BR = 40; | FiO2 = 30-40; | FiO2 = 30-40; |
| | FiO2 = 30-40; | FiO2 = 40-30; | FiO2 = 30-40; | FiO2 = 30-40; | FiO2 = 30-40; | Vdel = | Vdel = |
| | Vdel = | Vdel = | Vdel = | Vdel = | Vdel = | 5 ml; | 5 ml; |
| | 5 ml; | 5 ml; | 10 ml; | 30 ml; | 5 ml; | flow = | flow = |
| Time | flow = .5 L/min | flow = .5 L/min | flow = .5 L/min | flow = .5 L/min | flow = .5 L/min | 1 L/min | 5 L/min |
| 0 | 0.300011 | 0.399833 | 0.299947 | 0.300043 | 0.299889 | 0.300011 | 0.300089 |
| 1.00 | 0.299215 | 0.395067 | 0.300426 | 0.30073 | 0.300634 | 0.29919 | 0.325255 |
| 2.00 | 0.298623 | 0.393941 | 0.300725 | 0.301262 | 0.299471 | 0.301431 | 0.356951 |
| 3.00 | 0.300468 | 0.391891 | 0.30056 | 0.303474 | 0.29809 | 0.307779 | 0.377922 |
| 4.00 | 0.300775 | 0.391257 | 0.303689 | 0.305953 | 0.301957 | 0.309512 | 0.389504 |
| 5.00 | 0.302031 | 0.38877 | 0.30136 | 0.30677 | 0.301915 | 0.315724 | 0.392929 |
| 6.00 | 0.306689 | 0.38629 | 0.298887 | 0.309235 | 0.306758 | 0.324465 | 0.394496 |
| 7.00 | 0.311109 | 0.382054 | 0.300026 | 0.317203 | 0.308962 | 0.331977 | 0.392862 |
| 8.00 | 0.316073 | 0.371524 | 0.303994 | 0.320658 | 0.315336 | 0.341927 | 0.395584 |
| 9.00 | 0.322731 | 0.362666 | 0.310108 | 0.324641 | 0.320902 | 0.349178 | 0.399475 |
| 10.00 | 0.332193 | 0.360333 | 0.316429 | 0.330817 | 0.327037 | 0.354954 | 0.399702 |
| 11.00 | 0.341885 | 0.359144 | 0.324663 | 0.334608 | 0.331729 | 0.359272 | 0.400697 |
| 12.00 | 0.348825 | 0.350245 | 0.335529 | 0.343004 | 0.338817 | 0.367859 | 0.400167 |
| 13.00 | 0.352733 | 0.340902 | 0.344331 | 0.351919 | 0.344418 | 0.375256 | 0.3998 |
| 14.00 | 0.358411 | 0.33469 | 0.34836 | 0.360938 | 0.350779 | 0.381437 | 0.399594 |
| 15.00 | 0.367465 | 0.331315 | 0.354921 | 0.371246 | 0.354557 | 0.380556 | 0.40042 |
| 16.00 | 0.372674 | 0.326185 | 0.361406 | 0.379636 | 0.359411 | 0.378025 | 0.395219 |
| 17.00 | 0.373534 | 0.323427 | 0.368205 | 0.380761 | 0.361091 | 0.380544 | 0.392746 |
| 18.00 | 0.378429 | 0.319917 | 0.372608 | 0.382995 | 0.364546 | 0.388429 | 0.39104 |
| 19.00 | 0.382656 | 0.316281 | 0.376153 | 0.385669 | 0.371067 | 0.390386 | 0.391966 |
| 20.00 | 0.385744 | 0.309696 | 0.376743 | 0.38705 | 0.375005 | 0.388905 | 0.393127 |
| 21.00 | 0.386320 | 0.30689 | 0.380985 | 0.38547 | 0.375543 | 0.389443 | 0.394431 |
| 22.00 | 0.387645 | 0.310872 | 0.384559 | 0.38739 | 0.3786 | 0.390457 | 0.397938 |
| 23.00 | 0.386478 | 0.309744 | 0.384315 | 0.385677 | 0.381505 | 0.392341 | 0.396451 |
| 24.00 | 0.388219 | 0.310082 | 0.383796 | 0.3859 | 0.380195 | 0.390927 | 0.39588 |
| 25.00 | 0.388975 | 0.308916 | 0.387054 | 0.38787 | 0.380682 | 0.393864 | 0.394089 |
| 26.00 | 0.385936 | 0.30672 | 0.384204 | 0.387796 | 0.378965 | 0.393139 | 0.397215 |
| 27.00 | 0.385070 | 0.303732 | 0.386688 | 0.390132 | 0.376969 | 0.394375 | 0.398793 |
| 28.00 | 0.385717 | 0.303367 | 0.389892 | 0.39449 | 0.376479 | 0.396339 | 0.398044 |
| 29.00 | 0.387277 | 0.303108 | 0.392325 | 0.394347 | 0.383009 | 0.393793 | 0.397195 |
| 30.00 | 0.390118 | 0.304472 | 0.391711 | 0.392285 | 0.385854 | 0.389017 | 0.396688 |
| 31.00 | 0.390699 | 0.304861 | 0.391487 | 0.390168 | 0.388123 | 0.386432 | 0.395235 |
| 32.00 | 0.388523 | 0.306287 | 0.391328 | 0.391036 | 0.388983 | 0.388908 | 0.397169 |
| 33.00 | 0.389809 | 0.305668 | 0.389244 | 0.393922 | 0.389269 | 0.392722 | 0.399838 |
| 34.00 | 0.390700 | 0.304566 | 0.392614 | 0.395617 | 0.391185 | 0.393303 | 0.402126 |
| 35.00 | 0.392973 | 0.306325 | 0.393903 | 0.397594 | 0.390908 | 0.394895 | 0.402424 |

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for controlling an amount of oxygen in blood in a patient being ventilated by a medical ventilator, the method comprising:
monitoring an amount of oxygen in blood in a patient during ventilation on the medical ventilator;
monitoring privileged ventilator information, the privileged ventilator information is flow rate, compliance of patient circuit, and minute volume; and
controlling at least one of a specific oxygen percentage in a gas mixture supplied by the ventilator to the patient and a gas flow rate of the gas mixture supplied by the ventilator to the patient during ventilation based on the monitored amount of oxygen in the blood in the patient and the monitored privileged ventilator information.

2. The method of claim 1, wherein the privileged ventilator further comprises ideal body weight.

3. The method of claim 2, further comprising estimating circulatory time and lung washout time based on the ideal body weight.

4. The method of claim 1, wherein the step of controlling the at least one of the specific oxygen percentage in the gas mixture supplied by the ventilator to the patient and the gas flow rate of the gas mixture supplied by the ventilator to the patient during ventilation, comprises:
controlling the specific oxygen percentage in the gas mixture supplied by the ventilator by adjusting a gain coefficient of a controller utilizing a PID method based on the monitored amount of oxygen in the blood in the patient and the privileged ventilator information.

5. The method of claim 1, wherein the step of controlling the at least one of the specific oxygen percentage in the gas mixture supplied by the ventilator to the patient and the gas flow rate of the gas mixture supplied by the ventilator to the patient during ventilation, comprises:
utilizing fuzzy logic to automate an adjustment of the specific oxygen percentage based on the monitored amount of oxygen in the blood in the patient and the privileged ventilator information.

6. The method of claim 1, further comprising:
determining that the amount of oxygen in the blood in the patient during ventilation on the medical ventilator is at least one of above a preset high threshold and below a preset low threshold;
wherein the step of controlling the at least one of the specific oxygen percentage in the gas mixture supplied by the ventilator to the patient and the gas flow rate of the gas mixture supplied by the ventilator to the patient during ventilation, comprises:
changing the specific oxygen percentage in the gas mixture in preset increments until the amount of oxygen in the blood in the patient is between the preset high threshold and the preset low threshold.

7. The method of claim 1, further comprising controlling the washout time for an inspiratory limb of a patient circuit.

8. The method of claim 1, further comprising implementing a fast washout cycle by increasing flow to an appropriate higher value while opening both inspiratory and expiratory valves.

9. The method of claim 8, wherein the fast washout cycle reduces washout time by at least 25%.

10. The method of claim 8, wherein the fast washout cycle reduces washout time by at least 75%.

11. The method of claim 1, wherein the monitored amount of oxygen in the blood is SpO₂.

12. The method of claim 1, wherein the monitored amount of oxygen in the blood is PaO₂.

13. A medical ventilator system comprising:
a processor;
a patient circuit;
an oximeter connected to a patient being ventilated by the medical ventilation system and controlled by the processor, the oximeter is adapted to monitor a blood oxygen saturation level of the patient during ventilation by the medical ventilator system; and
an SpO₂ controller in communication with the processor and the oximeter and adapted receive the monitored blood oxygen saturation level from the oximeter, adapted to receive privileged ventilator information from the processor, and adapted to control at least one of a specific oxygen percentage and a flow rate of a gas mixture supplied to the patient during ventilation by the medical ventilator system based on the monitored blood oxygen saturation level of the patient and the privileged ventilator information,
wherein the privileged ventilator information is flow rate, compliance of a patient circuit, minute volume, and ideal body weight.

14. The medical ventilator of claim 13, further comprising a flow valve controlled by the processor, the flow valve is adapted to regulate a flow rate, a pressure, a volume, and gas concentrations of the gas mixture from a gas supply to the patient being ventilated by the medical ventilator system via the patient circuit.

15. The medical ventilator of claim 13, further comprising at least one sensor in the patient circuit controlled by the processor and adapted to monitor at least one of a respiratory rate, a tidal volume, or a compliance of the patient circuit.

16. The medical ventilator of claim 13, further comprising an operator interface in communication with the processor, the operator interface is adapted to receive user inputs and commands.

17. A method for controlling an amount of oxygen in blood in a patient being ventilated by a medical ventilator, the method comprising:
monitoring an amount of oxygen in blood in a patient during ventilation on the medical ventilator;
monitoring privileged ventilator information, the privileged ventilator information is flow rate, compliance of patient circuit, and minute volume;
detecting apnea in the patient based on the monitored amount of oxygen in the blood in the patient and the monitored privileged ventilator information; and
sending a few small breaths through the ventilator circuit to stimulate breathing in the patient.

18. The method of claim 17, wherein the monitored amount of oxygen in the blood is SpO₂.

19. The method of claim 17, wherein the wherein the monitored amount of oxygen in the blood is PaO₂.

20. A medical ventilator system comprising:
a processor;
a patient circuit;
a blood gas monitor connected to a patient being ventilated by a medical ventilator system and controlled by the processor, the blood gas monitor is adapted to monitor a partial pressure of oxygen in the patient during ventilation by the medical ventilator system; and
a PaO₂ controller in communication with the processor and the blood gas monitor and adapted to receive the monitored partial pressure of oxygen in the patient from the blood gas monitor, adapted to receive privileged ventilator information from the processor, and adapted to control at least one of a specific oxygen percentage and a flow rate of a gas mixture supplied to the patient during ventilation by the medical ventilator system based on the monitored partial pressure of oxygen in the patient and the privileged ventilator information,
wherein the privileged ventilator information is flow rate, compliance of a patient circuit, minute volume, and ideal body weight.

21. A computer-readable medium having computer-executable instructions for performing a method for controlling an amount of oxygen in blood in a patient being ventilated by a medical ventilator, the method comprising:
repeatedly monitoring an amount of oxygen in blood in a patient during ventilation on a medical ventilator;
repeatedly monitoring privileged ventilator information, the ventilator privileged information comprises flow rate, compliance of a patient circuit, and minute volume;
determining that a change in at least one of an oxygen percentage or flow rate is necessary based on the monitored amount of oxygen in the blood in the patient and the monitored privileged ventilator information; and
adjusting at least one of the oxygen percentage in a gas mixture and the gas flow rate of the gas mixture supplied by the ventilator to the patient during ventilation based on the monitored amount of oxygen in the blood in the patient and the monitored privileged ventilator information.

22. A medical ventilator system, comprising:
means for repeatedly monitoring oxygen saturation level of blood in a patient during ventilation on the medical ventilator;
means for repeatedly monitoring privileged ventilator information, wherein the privileged ventilator information comprises flow rate, compliance of a patient circuit, minute volume, and ideal body weight;
means for determining if a change in at least one of an oxygen percentage or flow rate is necessary based on the monitored oxygen saturation level of the blood of the patient and the monitored privileged ventilator information; and
means for adjusting at least one of the oxygen percentage in a gas mixture and the gas flow rate of the gas mixture supplied by the ventilator to the patient during ventilation based on the monitored oxygen saturation level of the blood of the patient and the monitored privileged ventilator information.
